(19) United States
(12) Patent Application Publication
(10) Pub. No.: US 2013/0295681 A1
(43) Pub. Date: Nov. 7, 2013

(54) DEVICE, SYSTEM AND METHOD FOR MEASURING HOMEOSTASIS, VITALITY, ENERGY AND STRESS IN AN INDIVIDUAL OR SUBSTANCE

(71) Applicant: Nader Butto, Petach Tikva (IL)

(72) Inventor: Nader Butto, Petach Tikva (IL)

(21) Appl. No.: 13/994,848

(22) PCT Filed: Dec. 15, 2011
(86) PCT No.: PCT/IB2011/055711

§ 371 Date: Jul. 2, 2013

Related U.S. Application Data
(60) Provisional application No. 61/423,547, filed on Dec. 15, 2010.

(51) Int. Cl. G01N 33/50

(52) U.S. Cl. CPC G01N 33/50
USPC 436/163; 422/68.1

(57) ABSTRACT

The present invention relates to a device, system and a method for evaluating, measuring and assessing vitality, energy and stress levels in an individual and/or substance, and in particular, to such a device, system and method in which personal assessment and decision support is provided based on the measured levels of vitality, energy and stress.

### FIELD OF THE INVENTION

The present invention relates to a device, system and a method for evaluating, measuring and assessing the state of wellbeing by measuring the stress level, and the energy resources (vitality) viability in an individual and/or substance (food), and in particular, to such a device, system and method in which personal assessment and decision support is provided based on the measured levels of vitality, energy and stress.

### BACKGROUND OF THE INVENTION

The constant pressure associated with living in a fast-paced world has created an environment where nearly everyone feels the effects of stress. Stress is a term used to describe the body and mind's response to any pressure that disrupts its normal balance.

Long standing maladaptive stress associated with chronic oxidative stress can become disabling may result in vital energy sources exhaustion.

Change, illness, injury or career and lifestyle changes, are common causes of stress. As a response, stress expresses itself as resistance, tension, strain or frustration that throws off our physiological and psychological equilibrium, keeping us out of homeostasis.

The goal of any system especially that of a biological system is to reach a balancing point or homeostasis. Homeostasis is one of the fundamental characteristics of living things to regulate its internal environment to maintain a stable, constant condition, by means of multiple dynamic equilibrium adjustments, controlled by interrelated regulation mechanisms.

The internal environment features body fluids include blood plasma, interstitial fluid and intracellular fluid which are considered the biological terrain that determines the normal function of the cells and maintain adequate energy reservoir. The interstitial fluid nourishes all the cells in our body and guide the delivery of electrolytes, minerals, enzymes, vitamins, hormones and water to the cells, and transports the substances and waste products that the cells release.

The internal environment and homeostasis are influenced by a myriad of factors like bad nutrition, emotional stress, oxidative stress, environmental pollution and others that can change the pH and the oxidative reductive potential of the blood and consume vital energy reserves.

The vital energy reserves contribute to the maintenance of equilibrium, homeostasis. The body is constantly trying to attain homeostasis, sometimes all it needs is a gentle push in the right direction. If the system does not succeed in reestablishing its balance due to excess of stress and to vital energy depletion, it may ultimately lead the system to stop functioning and disease will appear. If correct balance can be reached, the body's internal defense mechanism can begin the process of healing.

### SUMMARY OF THE INVENTION

In an attempt to reach equilibrium and homeostasis people employ a great variety of tools and spend many resources such as diets, homeopathic medicaments, herbal medicaments, drugs, exercise, psychologists, however none of these methods provide a user with a measureable, quantifiable means for reaching homeostasis.

There is an unmet need for, and it would be highly useful to have, a device, system and a method for evaluating the internal environment and assisting individuals in attempting to reach homeostasis by quantifying and/or measuring the state of an individual and/or substance in its contribution toward homeostasis. More specifically an aspect of the present invention provides for measuring vitality (energy resources), and stress level in an individual and measuring vitality and energy in substances for example foodstuffs) so as to map and or identify the specific food required to restore homeostasis.

Within the context of this application the term vitality refers to biological energy in the cells needed to transport ions and establish a transmembrane potential, and permit all physiological and metabolic processes. This energy drives from the ATP produced in the mitochondria, and it is considered to be the common currency of biological energy. The mitochondrial insufficiency, leads to oxidative stress and free radicals production. The vitality level can be measured by rH measurement which is derived from the measurement of Oxidation Reduction Potential (ORP) measured in millivolts (mv.) and pH in the venous blood.

The relative hydrogen or rH scale is a scale which measures true reducing power of simple compounds (very low molecular weight antioxidants) in aqueous solution. Unlike ORP, which yields a rough indicator of the amount of hydrogen reducing power available, but which is highly dependent upon the untoward and undesired effects of pH (e.g., the H+ ion, or hydrogen proton, which, when present in quantity, makes substances acidic), the rH yields a true index of hydrogen reducing power available, fully compensated for pH, to allow removal of all influence of the H+ ion (which makes a substance acidic if present in quantity.) The rH-value is obtained mathematically using pH, ORP, temperature, and is derived through the Nernst equation. It indicates the amount of electron potential in the fluid that is being tested.

The rH scale employs the same logarithmic Bar scale used to express gas pressure in terms of order of magnitude, and the rH scale runs from 0 to 42; with 28 being the mid-point. A value of rH under 28 is reduced, with a higher number of electron donors than acceptors.

The rH specifies the electrons available in a liquid for a particular pH value. This is the ability of a liquid to accept or release electrons, or to be alive. It represents the electrical energy of a liquid. A high number of available electrons in human cells indicate an optimum situation, signifying a healthy and productive Krebs cycle. In other words, the rH values show the concentration of electrons and their movement in the body. The rH value yields a hydrogen proton-unbiased measure of absolute reducing potential (Redox) of a substance; eliminating effects due to pH.

A preferred embodiment of the present invention provides a device, most preferably a non-invasive device, providing a scientific method for measuring the distress state of an individual based on at least one or more parameters for example including but not limited to HRV.

An optional embodiment provides for evaluating the oxidative stress of an individual or substance by measuring the redox potentials that may in turn provide useful information regarding the condition of body biochemistry, uncover nutritional imbalances, to overall health and state of bodily defense mechanism and/or energy viability.

Most preferably optional embodiment of the present invention provide for defining the extent and direction of the pathological deviation relative to optimal healthy values.

An optional embodiment of the present invention optionally and preferably provides for ascertaining the pathological deviation that may then provide for elucidating a corrective path to optionally and preferably restore the original optimal state of health of an individual.

An optional embodiment of the present invention provides for depicting the corrective path by elucidating and/or customizing stress reduction programs, nutritional, herbal and supplemental programs to address the source of the oxidative stress and track the health progress of an individual.

Within the context of this application the term Heart Rate Variability (herein referred to as ‘HRV’) may be measured, derived or determined with a plurality of optional sensors and/or techniques for example including but not limited to signals such as ECG, PPG, Pulse Oximeter, cardiac signal or the like or a combination thereof. Processing of HRV may optionally be facilitated by a plurality of optional methods as are known in the art such as spectral analysis, wavelet analysis, temporal analysis, nonlinear analysis, linear analysis or the like.

Within the context of this application the term Redox potential, rH2, rH2 and rH may be used interchangeably to refer to the reducing strength of a substance. For example, ideal blood rH2 values should be between 23 and 24. Readings greater than 24 reflects increasing oxidative stress. Oxidative stress is caused by mitochondrial insufficiency, but antioxidant deficiency is often a contributing factor.

Nutritional deficiencies, stress, over stimulation of the immune system, lack of exercise, exposure to radiation, and toxicity can all lead to higher than optimum levels. The individual can then be more prone to disease and premature aging.

An optional embodiment of the present invention provides a device, system and method for determining, measuring, evaluating and/or scoring a substance relative to its contribution to an individual's homeostasis. Optionally and preferably a substance (for example including but not limited to food, foodstuffs) is scored relative to its pH and rH (most preferably identifying the level of vitality and energy in the foodstuffs) in order to identify if such food contributes or hinders an individuals state of homeostasis.

Optionally a substance's contribution to the homeostatic flux of an individual may be measured with at least one or more sensors, most preferably to measure the pH and Redox (rH) of the substance being measured. Optionally and preferably pH and Redox readings are then processed to produce a score reflective of the substances vitality, energy states and its code, according to an optional embodiment of the present invention provides. Optionally, the substance code will be fit to the human code determined by the facial feature or by other means.

Optionally and preferably the substance may be liquefied prior to measuring. The measured substance may for example comprise foodstuff, clothing, biological fluids, or the like.

An optional embodiment of the present invention provides a device, system and method for evaluating and/or determining an individual's relative state of homeostasis. Optionally and preferably the homeostatic state is provided relative to an individual's human code and/or universal code that optionally and preferably serves as a the homeostatic goal.

Optionally an individual's instant state of homeostatic with respect to a homeostatic goal, optionally and preferably the individual's human code or universal code may be determined by a device, system and method of the present invention. An individual's levels of vitality, energy, and stress levels reflective of the homeostatic state are determined by measuring and processing the HRV of the individual along with an individual's pH and Redox of at least one or more bodily fluid, for example including but not limited to saliva, urine, blood, seminal fluid, plasma, feces, tears, sweat, vaginal fluid, cerumen, breast milk, gastric mucosa, gastric fluids, gastric flora, sebum, oral flora, oral mucosa. Optionally measuring HRV, pH and Redox is provided by at least one or more sensors.

Balance and homeostasis derives from the result of the activities of the opposing forces and depends in one hand on balance between the sympathetic and parasympathetic systems, and the other hand on the free radicals and antioxidants. The values of these measurements are necessary and sufficient to determine any general biological condition of homeostasis, wellbeing, health, illness, and to evaluate the influence of environmental factors such as emotional stress, drinking water, food etc. and the real value of different therapies.

Use of offline data for example including but not limited to geographic location, universal code, human code, personal data, further observations may for example be based on and include but is not limited to data entered manually based on a the subject's personal data, questionnaire, or task performance results, and observed data, or the like.

Optionally a device according to optional embodiments may provide for abstracting an index for a plurality of optional substances for example foodstuff. For example, a device may be utilized to provide an index indicative of the homeostatic value of foodstuff such as tea, soup or the like providing an indication of its homeostatic value to an individual in his attempt to reach homeostasis.

An optional embodiment of the present invention provides a device for indexing and/or scoring and/or evaluating a substance based on at least two or more parameters for example including but not limited to pH and redox (rH2) levels.

Optionally the pH and redox parameters may be measured with at least one sensor.

Optionally the substance may for example include but is not limited to a medicament, biological fluid, foodstuff, fluids, tea, juices, edible solids, liquefied edible solids, or the like.

Optionally a biological fluid may be chosen from the group for example including but not limited to saliva, urine, blood, seminal fluids, plasma, feces, tears, sweat, vaginal fluid, cerumen, breast milk, gastric fluids, gastric flora, sebum, oral flora, oral mucosa, or the like.

Optionally a medicament may for example include but is not limited to a drug, traditional medicament, orthodox medicament, chemical, herbal medicine, homeopathic substance, naturopathic substance, or any combination thereof or the like.

Optionally the process of evaluating a substance provides for abstracting an individualized assessment relating to and/or associated with the substance.

An optional embodiment of the present invention provides a method for indexing and/or scoring and/or evaluating an optional substance based on at least two parameters for example including but not limited to pH and redox, the method comprising:

a. Liquefying a substance to be indexed/scored/evaluated; and

b. Associating the liquefied substance with at least one sensor for determining at least two or more parameters including pH and Redox; and

c. Processing the at least two or more parameters including pH and Redox for indexing and/or scoring and/or evaluating the liquefied substance.

Optionally the substance may for example include but is not limited to a medicament, biological fluid, foodstuff, fluids, tea, juices, edible solids, liquefied edible solids, or the like.

Optionally the biological fluid may be chosen from the group for example including but not limited to saliva, urine, blood, seminal fluids, plasma, feces, tears, sweat, vaginal fluid, cerumen, breast milk, gastric fluids, gastric flora, sebum, oral flora, oral mucosa, or the like.

Optionally a medicament may for example include but is not limited to a drug, traditional medicament, orthodox medicament, chemical, herbal medicine, homeopathic substance, naturopathic substance, or any combination thereof or the like.

An optional embodiment of the present invention provides a device for abstracting and individual assessment comprising the level of stress, vitality and energy of an individual based on measured parameters including HRV, redox, and pH, the device comprising:

a. At least one or more sensor for obtaining the HRV, redox and pH measurements from a subject; and

b. a processor for processing the measurements and abstracting the individual assessment.

Optionally the device may further comprise a display for displaying the individualized assessment.

Optionally the device may further comprise a communication module for communicating the individualized assessment.

Optionally the redox and pH measurements may be obtained from a bodily fluid and/or biological fluid of an individual to be assessed.

Optionally the biological fluid and/or bodily fluid may be chosen from the group for example including but not limited to saliva, urine, blood, seminal fluids, plasma, feces, tears, sweat, vaginal fluid, cerumen, breast milk, gastric fluids, gastric flora, sebum, oral flora, oral mucosa, or the like.

Optionally the HRV may be obtained from a subject with at least one or more sensors for example including but not limited to ECG, pulse-oximeter, PPG or the like.

Optionally individual assessment provides for a decision support relating to an individual's personal aspects for example including but not limited to food, drugs, medicaments,

Optionally a diagnostic assessment may be provided with respect to an individual's human code.

Optionally an individual assessment may be provided for individual personality assessment with respect to an individual human code.

Optionally an individual assessment may be provided for interpersonal assessment.

An optional embodiment of the present invention provides a method for abstracting an individual's human code utilizing a device according to an optional embodiment of the present invention, the method comprising:

a. Providing measurements for redox, pH and HRV data; and

b. processing the measurement to abstract an individual's human code.

An optional embodiment of the present invention provides a method for diagnostic assessments, comprising: determining the human code; and abstracting diagnosis relative to the human code.

Optionally an individual assessment may be provided in the form of a dietary diagnosis.

Optionally the decision support module may select at least one or more treatment modalities for example including but not limited to dietary, conventional medicine, traditional medicine, acupuncture, aromatherapy, music therapy, psychological, physiological, energy, or the like or any combination thereof.

Optionally personal parameters may be used to abstract an individual's human code.

Optionally the personal parameters may for example include but are not limited to at least one or more of subject's name, subject's mother's name, a subject's date of birth or the like.

Optionally the system according to an optional embodiment of the present invention may be adapted to provide a user's educational assessment.

Optionally the system according to an optional embodiment of the present invention may be adapted for converting the human code to the universal code.

Unless otherwise defined the various embodiment of the present invention may be provided to an end user in a plurality of formats, platforms, and may be outputted to at least one of a computer readable memory, a computer display device, a printout, a computer on a network or a user.

Unless otherwise defined, all technical and scientific terms used herein have the same meaning as commonly understood by one of ordinary skill in the art to which this invention belongs. The materials, methods, and examples provided herein are illustrative only and not intended to be limiting. Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Although the present invention is described with regard to a “computer” on a “computer network”, it should be noted that optionally any device featuring a data processor and/or the ability to execute one or more instructions may be described as a computer, including but not limited to a PC (personal computer), a server, a minicomputer, a cellular telephone, a smart phone, a PDA (personal data assistant), a pager. Any two or more of such devices in communication with each other, and/or any computer in communication with any other computer may optionally comprise a “computer network”.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A-D are schematic illustrative block diagrams of optional non limiting embodiments of a device according to the present invention;

FIG. 2 is a schematic illustrative block diagram of an exemplary non limiting system according to the present invention;

FIG. 3 is an exemplary method according an optional embodiment of the present invention for measuring the vitality, energy and stress of a substance; and

FIG. 4 is an exemplary method according to an optional embodiment of the present invention for assessing an individual's instant homeostatic state.

### DESCRIPTION OF THE PREFERRED EMBODIMENTS

The present invention is of a device, system and a method for evaluating, measuring and assessing the homeostatic state of a substance or individual by measuring at least two or more parameters that are processed to identify the levels of vitality, energy and stress levels in an individual and/or substance. The system may further provide a decision support system where for assessing the interaction and interdependence between individual and the substance.

The principles and operation of the present invention may be better understood with reference to the drawings and the accompanying description. The following reference labels listed below are used throughout the drawings to refer to objects having similar function, meaning, role, or objective.
100, 101 device for measuring homeostatic state;
102 communication module;
104 processor;
106 display;
110 sensor;
112 pH sensor;
114 HRV sensor;
116 Redox sensor;
120 processing center;
200 system;
202 server;
204 communication medium.

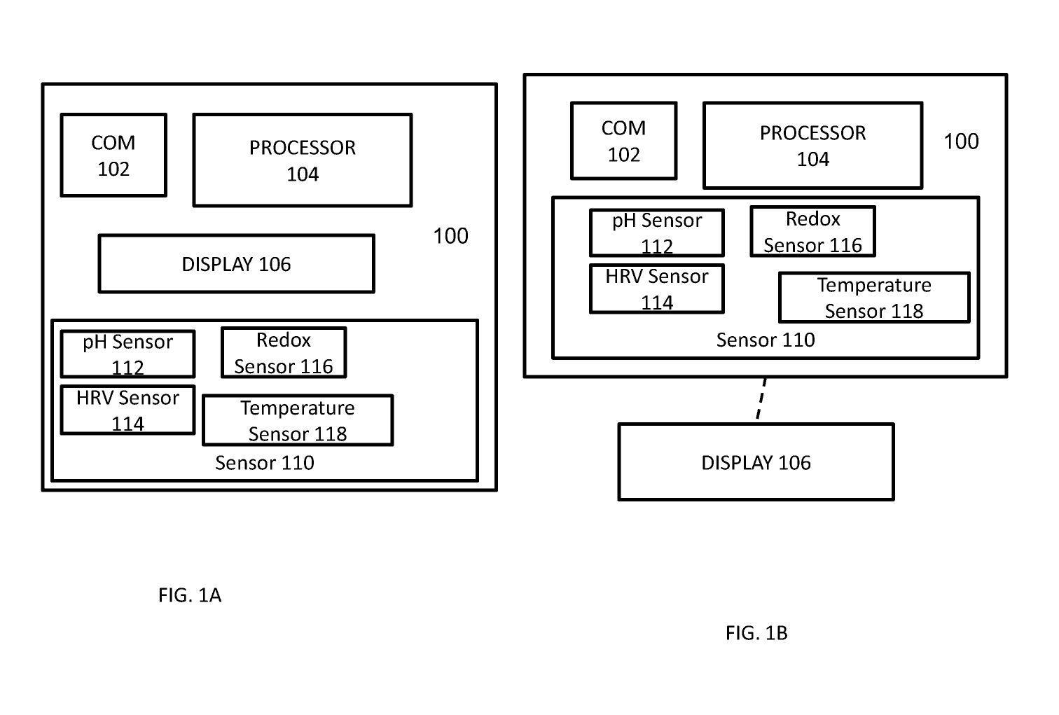

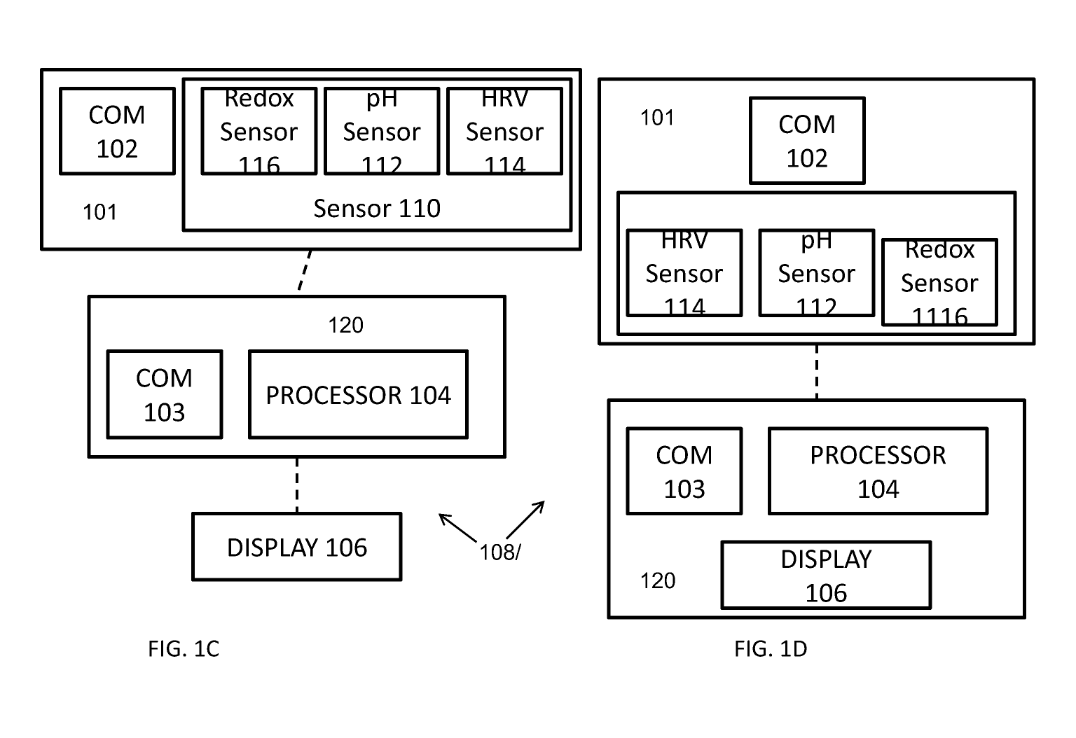

Referring now to the drawings, FIG. 1A shows a device 100 for assessing the homeostatic state of an individual or substance according to the present invention. Device 100 comprises sensor 110, communication module 102, processor 104 and display. Most preferably device 100 provides for measuring a substance in a liquefied form. Device 100 provides form measuring a liquid in various forms for example including but not limited to a biological fluid, bodily fluid, foodstuff or the like. Optionally device 100 provides for measure substances that are liquefied optionally in a solvent. For example, foodstuff in solid state may be liquefied in a solvent, for example water, alcohol, oil or the like.

Optionally sensor 110 provides a sensor capable of measuring at least one or more signals for example including but not limited to pH, redox and HRV or the like. Optionally sensor 110 provides for a sensor array comprising at least one or more sensors for measuring pH, redox or an individual or substance. For example an individual's pH and redox may be determined based on the measured pH and redox of at least one or more biological fluid attained from the individual. Optionally sensor 110 further comprises a sensor for measuring HRV of an individual, optionally HRV may be attained by processing the cardiac signals of an individual for example including but not limited to ECG, PPG, pulse-oximeter or the like.

Optionally communication module 102 is provided for communicating to a processor 104 and/or display 106 or an auxiliary device (not shown) the measured parameters with sensor 110-116 for example including but not limited to pH, redox and/or HRV as substance or individual.

Optionally processor 104 is provided for processing the measured signals provided by sensor 110-116. Optionally and preferably processor 104 provides for processing data obtained from sensor 110-116 via communication module 102. Optionally and preferably the processing provided by processor 104 provides an analysis where at the measured pH, Redox, and HRV are transformed and/or converted to provide an index, index, score or the like evaluation indicative and/or reflective of overall homeostasis of the measured substance and or individual by indicating the vitality, stress and energy levels of the measured substance and/or individual.

Optionally display 106 is provided for displaying the measured parameters by sensors 110-116 or the processed data provided by processor 104. Display 106 may optionally be provided in the form of an LCD display, LCD screen, LED array, a patient monitor or the like means for displaying.

FIG. 1B depicts an optional configuration of device 100 described in FIG. 1A, wherein display 106 is provided in the form of an external or auxiliary display device that may be coupled to device 100 optionally comprising communication module 102, sensors 110-116, and processor 104.

FIG. 1C depicts a further optional configuration of a system 108 according to an optional embodiment of the present invention comprising device 101, processing center 120 and display 106. Device 101 comprises sensors 110-116 and communication module 102, similar to that provided by device 100. Device 101 provides an optional configuration of device 100, providing for measuring pH, redox, HRV of a substance and/or individual for abstracting the vitality, stress, and energy levels of the measured substance and/or individual for assessing the homeostatic state. Device 101 optionally comprises a sensor 110-116 providing measurement of pH, redox and HRV that may be processed with an auxiliary higher processing device 120 for example in the form of a server, computer, mobile telephone, minicomputer comprising a processor 104. Optionally processing center 120 further comprises a communication module 103 for communicating with communication module 102 and display 106.

FIG. 1D provides an optional configuration of system 108 depicted in FIG. 1C wherein processing center 120 comprises an internal display 106.

Optionally processor 104 and/or higher processing center 120 provide for analyzing and processing the parameters and measurements provided by sensors 110-116 and transforming them to an evaluation of the homeostatic state of the measured substance and or individual by indicating the stress, vitality and energy levels based on the measured parameters including pH, redox, and HRV, optionally this may be provided in the form of a score, Optionally processors 104, 120 may further provide features utilized to optionally abstract the human code or universal code associated with the substance and/or individual being measured. Optionally the processed information indicative to the stress, vitality, and energy levels may provide for medical diagnosis, interpersonal advice, job placement analysis, educational analysis or the like. Optionally, the human code may be converted to another code for example including but not limited to the universal code.

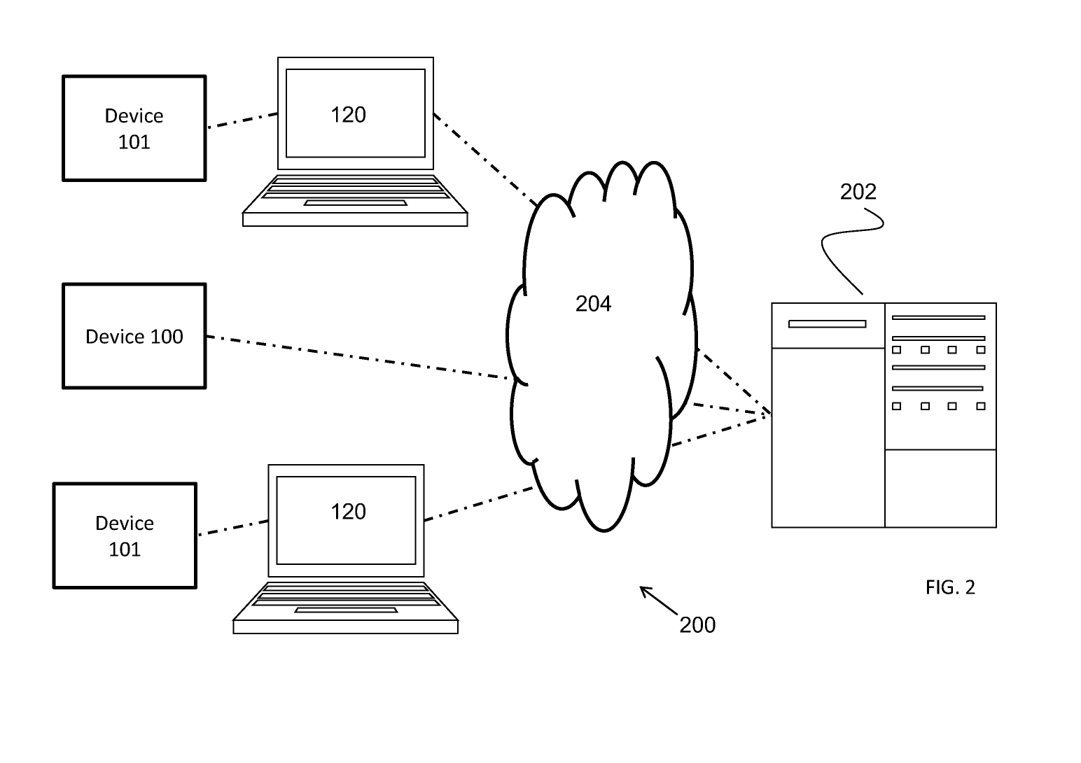

FIG. 2 provides an optional embodiment of a system 200 according to the present invention for depicting the homeostatic value of a substance and/or individual. System 200 may comprise a plurality of devices 100 and/or 101, 108 as depicted in FIGS. 1A-D, that are connected to a server 202 over a communication medium 204 for example including the internet, intranet or the like. Optionally system 200 provides a configuration of a call center or processing center that is able to process a plurality of substances and/or individuals for providing their homeostatic state relative to a database and or knowledgebase (not shown) in server 202. Optionally call center may provide a centralized location for example in the form of a website connected to a plurality of devices over an internet connection 204 adapted to providing decision support relating to the homeostatic state associated with the devices connected thereto. Optionally server 202 may be provided in the form a social network for identifying the homeostatic state of a substance or individual.

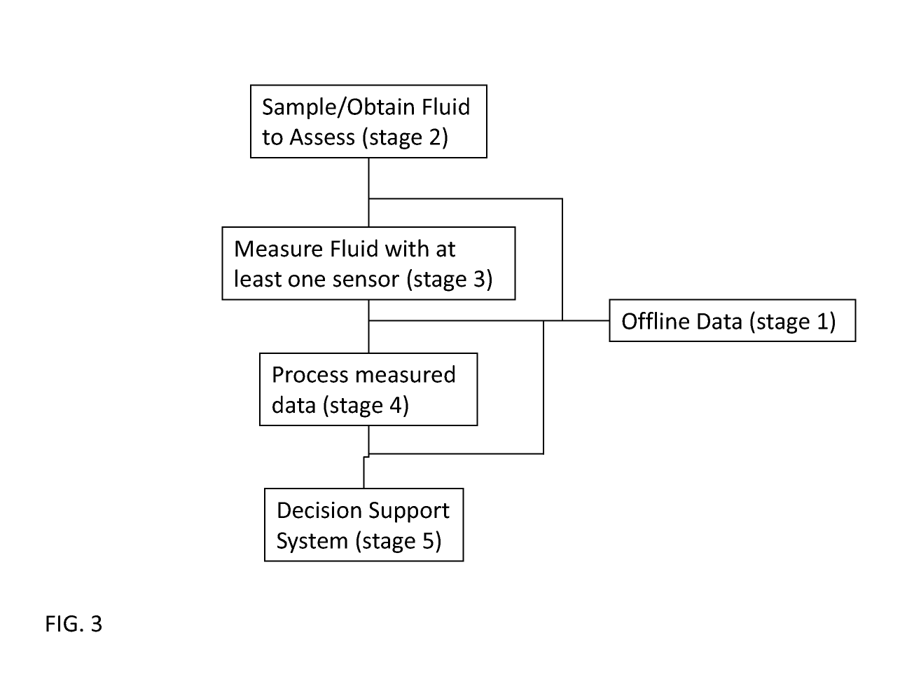

FIG. 3 shows a flowchart of an exemplary method according to the present invention for evaluating and or scoring a substance to determine its homeostatic contribution to an individual in measuring the vitality, energy, stress provided by the measured substance by measuring the pH, redox of the liquefied substance. Optionally the measured substance may for example include but is not limited to a bodily fluid, biological fluid, foodstuff, drug, traditional medicament, orthodox medicament, chemical, herbal medicine, homeopathic substance, naturopathic substance, fluids, tea, juices, edible solids, liquefied edible solids, or the like. Optional substances may include solids that may be liquefied with a solvent for example water, acid, alcohol, or the like solvents.

In stage 2 a sample of the substance to be measured is obtained. For example if a bodily fluid is to be evaluated it is extracted for example saliva or blood is obtained from an individual. Alternatively if a foodstuff is to be measured, for example a tea, the tea is prepared and a portion of it is set aside for evaluation and/or measurement. Optionally solid substances for example in the form of solid foods they are liquefied and sampled.

Next in stage 3 the sampled and/or liquefied substance is measured with the device 100, 101 of the present invention comprising at least one or more sensor 110-116. Most preferably sensors 110-116 provide for measuring the pH and redox of the sampled substance.

Next in stage 4, the measurements obtained in stage 2 are then communicated with communication module 102 to a processor or processing center 104, 120, 202 for analyzing and process the measured values of the pH and Redox of the sampled substance. Most preferably the processor then performs a transformation of the measured data converting it to indentify the substances value in terms of vitality, stress, and energy. Most preferably the evaluation of vitality, stress, and energy may then be utilized to indicate to an individual the contribution of the evaluated substance to their homeostatic state.

Next in stage 5 the processed scored substance is evaluated with the processors 104 or 120, 202 for abstracting a decision support or advice with respect to the measured substance and its homeostatic contribution to an individual.

Optionally in stage 1, offline data obtained relating the substance being measured or the individual for which it is intended is entered.

Optionally such offline data may be utilized at any point throughout the method as depicted in FIG. 3.

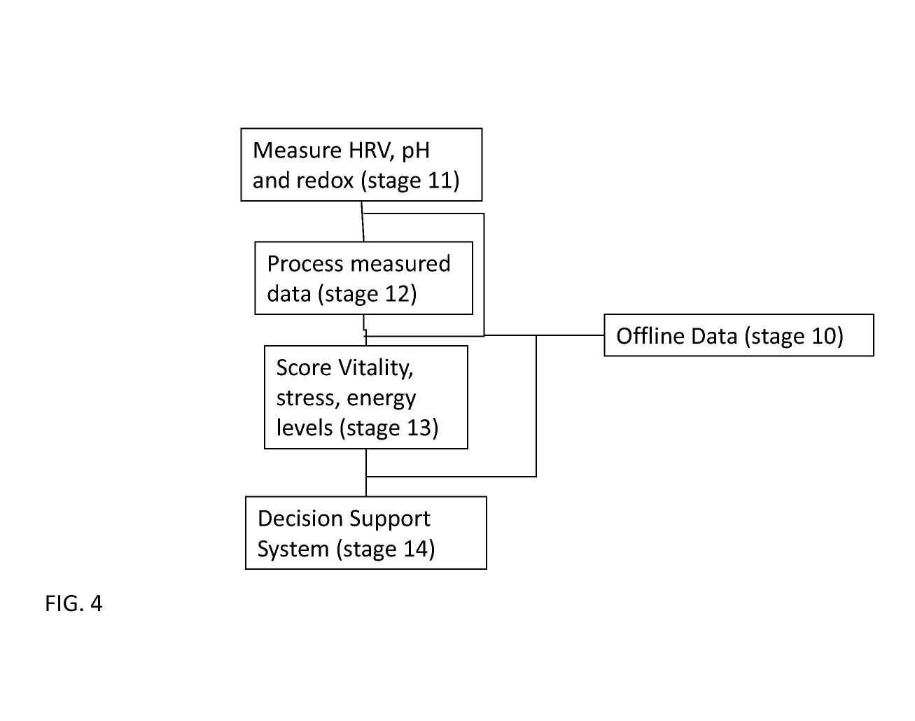

FIG. 4 shows a flowchart of an exemplary method according to the present invention for evaluating and or scoring an individual to determine the individual's homeostatic state. Most preferably the individual's homeostatic stat is determined in terms of a score measuring the vitality, energy, stress of the individual provided by determining the individual's HRV provided by a cardiac signal taken from the individual that is coupled with a measure at least one bodily fluid of the individual for assessing the individual's pH and redox of the bodily fluid. Optionally the measured bodily fluid or biological fluid may for example include but is not limited to a bodily fluid, biological fluid, saliva, urine, blood, seminal fluids, plasma, feces, tears, sweat, vaginal fluid, cerumen, breast milk, gastric fluids, gastric flora, sebum, oral flora, oral mucosa or the like.

In stage 11 a sample of the bodily fluid is obtained from an individual, and the cardiac signals from the individual are assessed to provide the individual's HRV. Optionally the sampled fluid and HRV values are measured with device 100, 101 of the present invention comprising at least one or more sensor 110-116. Most preferably sensors 110-116 provide for measuring the HRV, pH and redox.

Next in stage 12, the measurements obtained in stage 11 are then communicated with communication module 102 to a processor or processing center 104, 120, 202 for analyzing and process the measured values of the HRV, pH and Redox. Next in stage 13 processor 104, 120, 202 then performs a transformation of the measured data converting it to indentify the substances value in terms of vitality, stress, and energy. Most preferably the evaluation of vitality, stress, and energy may then be utilized to indicate to an individual the contribution of the evaluated substance to their homeostatic state.

Next in stage 14 the levels of vitality, stress, and energy associated with the individual are utilized to abstract a decision support and/or advice with respect to homeostatic state of the individual. Optionally the measured state is compared to an individual's universal code and/or human code.

Optionally in stage 10, offline data obtained relating the individual, for example historical data, is obtained. Optionally such offline data may be utilized at any point throughout stages 11-14 the method as depicted in FIG. 4.

### EXAMPLE

For example a person experiencing stress and therefore in a homeostatic flux, is prescribed a given homeopathic medicament to relieve the stress. Prior to ingesting the prescribed homeopathic medicament the person is evaluated for his homeostatic state according to the present invention as described in FIG. 4 while the medicament is separately evaluated as depicted in FIG. 3. Once both the individual is evaluated and the medicament is evaluate the decision support system may be further utilized to determine if indeed the medicament will contribute to the improving the homeostatic state off the individual or if the medicament will place the individual in a deeper state of homeopathic flux.

Optionally the homeostatic flux of the individual may be defined in accordance with the human code or universal code of the individual. Optionally, the homeostatic state determined by the device system and method of the present invention provides for comparison to an individual's homeostatic target that is optionally and preferably defined by the individual's human code and universal code.

While the invention has been described with respect to a limited number of embodiments, it will be appreciated that many variations, modifications and other applications of the invention may be made.

1. A device for evaluating a substance based on at least two parameters including pH and redox values.

2. The device of claim 1 wherein said substance is a biological fluid.

3. The device of claim 2 wherein said biological fluid is chosen from the group consisting of saliva, urine, blood, seminal fluids, plasma, feces, tears, sweat, vaginal fluid, cerumen, breast milk, gastric fluids, gastric flora, sebum, oral flora, oral mucosa.

4. The device of claim 1 wherein said substance is a foodstuff.

5. The device of claim 4 wherein said foodstuff is chosen from the group consisting of: fluids, tea, juices, edible solids, liquefied edible solids.

6. The device of claim 1 wherein said substance is a medicament.

7. The device of claim 6 wherein said medicament is chosen from the group consisting of a drug, traditional medicament, orthodox medicament, chemical, herbal medicine, homeopathic substance, naturopathic substance.

8. The device of claim 1 wherein said evaluation provides for abstracting an individualized assessment.

9. The device of claim 1 wherein said parameters are measured with at least one sensor.

10. The device of claim 1 wherein said parameters are measured with at least two sensors.

11. A method for evaluating a substance based on at least two parameters including pH and redox, the method comprising:
a. Liquefying a substance to be evaluated
b. Associating said liquefied substance with at least one sensor for determining at least two or more parameters including pH and Redox;
c. Processing said at least two or more parameters including pH and Redox for evaluating said liquefied substance.

12. The method of claim 11 wherein said substance is a biological fluid.

13. The method of claim 12 wherein said biological fluid is chosen from the group consisting of saliva, urine, blood, seminal fluids, plasma, feces, tears, sweat, vaginal fluid, cerumen, breast milk, gastric fluids, gastric flora, sebum, oral flora, oral mucosa.

14. The method of claim 11 wherein said substance is a foodstuff.

15. The method of claim 14 wherein said foodstuff is chosen from the group consisting of: fluids, tea, juices, edible solids, liquefied edible solids.

16. The method of claim 11 wherein said substance is a medicament.

17. The method of claim 16 wherein said medicament is chosen from the group consisting of a drug, traditional medicament, orthodox medicament, chemical, herbal medicine, homeopathic substance, naturopathic substance.

18. A device for abstracting an individual assessment comprising the level of stress, vitality and energy of an individual based on measured parameters including HRV, redox, and pH, the device comprising:
a. At least one or more sensor for obtaining said HRV, redox and pH measurements from a subject; and
b. a processor for processing said measurements and abstracting said individual assessment.

19. The device of claim 18 further comprising a display for displaying said individualized assessment.

20. The device of claim 18 further comprising a communication module for communicating said individualized assessment.

21. The device of claim 18 wherein said redox and pH are obtained from a bodily fluid or biological fluid of said individual.

22. The device of claim 21 wherein said bodily fluid or biological fluid is chosen from the group consisting of saliva, urine, blood, seminal fluid, plasma, feces, tears, sweat, vaginal fluid, cerumen, breast milk, gastric mucosa, gastric fluids, gastric flora, sebum, oral flora, oral mucosa.

23. The device of claim 18 wherein said HRV is obtained from a subject with at least one or more sensors chosen from the group consisting of ECG, pulse-oximeter, PPG.

24. The device of claim 18 wherein said individual assessment provides decision support relating to an individual's personal aspects for example including but not limited to food, drugs, medicaments.

25. The device of claim 18 adapted for diagnostic assessment with respect to an individual human code.

26. The device of claim 18 wherein said individual assessment is provided for individual personality assessment with respect to an individual human code.

27. The device of claim 18 adapted for interpersonal assessment.

28. A method for abstracting an individual's human code utilizing the device of claim 18, the method comprising:
a. Providing measurements for redox, pH and HRV data;
b. processing said measurement to abstract an individual's human code.

29. A method according to claim 28 adapted for diagnostic assessments, further comprising:
a. determining the human code
b. abstracting diagnosis relative to the human code.

30. The method of claim 29 wherein the diagnosis is a dietary diagnosis.

31. The method of claim 29 wherein a decision support module selects at least one or more treatment modalities chosen from the group consisting of dietary, conventional medicine, traditional medicine, acupuncture, aromatherapy, music therapy, psychological, physiological, energy.

32. The system of claim 1 wherein personal parameters are used to abstract an individual's human code.

33. The system of claim 16 wherein said personal parameters comprise subject's name, subject's mother's name, a subject's date of birth.

34. The system of claim 1 adapted for educational assessment.

35. The system of claim 1 adapted for converting the human code to the universal code.
